(19) United States
(12) Patent Application Publication
(10) Pub. No.: US 2004/0106201 A1
(43) Pub. Date: Jun. 3, 2004

(54) Method and device for evaluation of chemical reactions

(76) Inventors: Helmut Blum, Duesseldorf (DE); Wolf-Dieter Herberg, Duesseldorf (DE); Ludger Buetfering, Nideggen (DE); Holger Wildt, Duesseldorf (DE)

Correspondence Address: HENKEL CORPORATION, THE TRIAD, SUITE 200, 2200 RENAISSANCE BLVD., GULPH MILLS, PA, 19406, US

(21) Appl. No.: 10/399,831

(22) PCT Filed: Oct. 13, 2001
(86) PCT No.: PCT/EP01/11855

(30) Foreign Application Priority Data
Oct. 23, 2000 (DE) 10052511.3

(51) Int. Cl. G01N 33/00

(52) U.S. Cl. 436/34; 436/37; 422/68.1

(57) ABSTRACT

Disclosed is a system for monitoring chemical reactions, especially for detecting exothermic chemical reactions, comprising a reaction device consisting of a plurality of spatially separated reaction chambers for receiving reaction mixtures, and a dosing device for feeding reaction components of the reaction mixtures into the reaction chambers. The inventive system also comprises at least one sensor device which is sensitive to thermal radiation in order to detect the thermal radiation emitted by the reaction mixtures in the reaction chambers.

The present invention relates to a system for monitoring chemical reaction processes, in particular for recording exothermic chemical reaction processes, and to the use of such a system or of a sensor unit sensitive to thermal radiation and to a method for monitoring a multiplicity of chemical reaction mixtures.

The present invention furthermore relates to the field of combinatorial chemistry, in particular to a method and an apparatus for regulating and, where appropriate, controlling exothermic reaction processes, preferably in “screening” methods.

High efficacy, efficient cost-effective ingredients, and rapid market-readiness are nowadays the result-oriented parameters for central research projects. In research, new technologies, methods, formulations and product concepts are developed for future fields of business. One of these new concepts is “combinatorial chemistry” or “combinatorial synthesis”. Here, every day numerous series of experiments are carried out in parallel and in an automated manner, rapidly and in a miniaturized format so that said experiments require only very small amounts of components.

While previously in chemistry in particular organic synthesis endeavored to produce individual target compounds of a defined structure with very high selectivity and high yields and then subject them to a screening, combinatorial synthesis turns this principle on its head. Now it is the aim to synthesize simultaneously a plurality of compounds of defined structure from a number of structurally similar starting compounds. The basic idea here is that conventional rational synthesis can, with respect to the production of lead substances, no longer keep up with the performance of the available assay systems which are capable of testing thousands of new compounds per day for their effectiveness and efficiency. While combinatorial synthesis was initially applied mainly in the field of pharmaceutical-medical chemistry, it is now also widely used in other industrial fields of chemistry. The methods of combinatorial chemistry make it possible to rapidly check a plurality of compounds with the aid of the modern assay methods, thereby comparatively easily filtering out the active compounds. Disadvantageously, however, it may be possible that only very active substances are recognized. The principle of combinatorial synthesis is very simple: instead of reacting compound A with compound B to give the new compound AB, compounds A1-m are reacted with B1-n to give A1-nB1-m, where m and n are integers, it being possible to produce any combinations.

The dimensions opened up by combinatorial chemistry in the search for “hits”, i.e., in other words, new, efficient compounds and substance combinations detected, for example, within a series of experiments, can be illustrated by a simple example: around 20 million organic compounds are known to contain the universally present elements carbon (C), oxygen (O), hydrogen (H), sulfur (S) and/or nitrogen (N). It is theoretically possible to produce 1063 compounds from up to thirty atoms of said elements with the aid of combinatorial chemistry. If only one gram of each compound were produced, even the mass of the universe would be negligible in comparison therewith. This example indicates that it is impossible for the researcher to develop and find innovative chemical compounds and products merely through knowledge or understanding, taking into account all sensible possibilities. Previous research has been characterized by intuition, luck, trial and error, and also by lengthy and costly series of experiments. This does not necessarily always involve completely new compounds but likewise improving known syntheses or reducing the use of expensive raw materials with identical quality of the final product. In the automated experiment arrangements, this lengthy routine and precision work is nowadays carried out by a robot.

For further details of combinatorial chemistry, reference is made to the following prior art whose contents are hereby included with reference: Römpp Lexikon Chemie, 10th edition, volume 3, 1997, p. 2217, keyword “kombinatorische Synthese” [combinatorial synthesis] and volume 5, 1998, p. 4025, keyword “Screening” and Nachr. Chem. Tech. Lab. 44 (1996) No. 12, pp. 1182-1188 and Nachr. Chem. Tech. Lab. 45 (1997) No. 2, pp. 157-159; Chemical Reviews, volume 97, No. 2, March/April 1997, pp. 347/348; Angew. Chem. 1996, 108, No. 11, pp. 1235-1237 and Angew. Chem. 1997, 109, No. 8, pp. 857-859.

The multiplicity of samples obtained in combinatorial synthesis makes it necessary to study said samples systematically. This systematic examination of samples of natural or synthetic origin by suitable systems for the presence of low or high molecular weight substances having particular properties is referred to as screening. In industrial pharmaceutical research in particular, but also in agriculture, food technology and synthetic chemistry in other industrial fields of chemistry, screening is an important means in order to be able to access new or improved products or production methods. Success criteria for the results obtained in a screening are the selection or assay systems used. They need to be selective and as unambiguous as possible, with respect to their meaningfulness, easy to manage, rapid to carry out and have good reproducibility. With regard to the increasing number of test samples which in the meantime, for example in drug screening, come to several hundred thousands per test and year, and rising further, automation and miniaturization become increasingly important. Thus the spectrum of specialized disciplines needing to work together in screening is extended further. A high throughput of test compounds and test combinations and evaluation thereof are also referred to by the term “high throughput screening” (HTS). For further details, reference may be made to the literature cited above.

However, one difficulty in the conventional screening systems of the prior art is to find suitable systems, in particular assay systems, which selectively find the active compounds or compositions among a multiplicity of samples to be studied. The development of a suitable assay system is often very time-consuming and costly.

It is the object of the present invention to provide a system for monitoring chemical reaction processes, in particular for recording exothermic chemical reaction processes, which is easy to handle, and to indicate a method for monitoring chemical reaction processes, in particular for a multiplicity of chemical reaction mixtures.

Another object of the following invention is to check substances arising on a huge scale, such as those arising, for example, by combinatorial techniques, in a short period of time, in particular thermocurveically, and to indicate in particular a system, a use and a method which, with relatively low complexity and in a relatively simple, secure manner, enable preferably automated monitoring of, in particular, a multiplicity of chemical reaction processes for detecting exothermicity. In particular, it is intended to use such a system in combinatorial techniques, in particular in automated screening methods for selective detection of active compounds or compositions.

According to the proposal, the above object is achieved by a system as claimed in claim 1, a use as claimed in claims 28 and 35 or a method as claimed in claim 38. Advantageous developments are subject matter of the dependent claims.

A principal idea of the present invention is to provide for a heat-sensitive sensor, in particular an IR camera or the like, in order to record the thermal radiation emitted by reaction mixtures. Thus it is possible, in a simple, cost-effective manner, to detect exothermicity.

“IR” here means infrared radiation. Accordingly, an IR camera means a camera sensitive to thermal radiation. According to one development, the camera is also able to provide other optical signals, for example regarding a color change of reaction mixtures, the occurrence of bubbles (e.g. during boiling) or the like.

The term “detection of exothermicity” primarily means that the occurence of exothermicity, i.e. the exothermic course of chemical reactions, is detected and can be displayed accordingly and data relating thereto can be output. Preferably, the term should be interpreted broadly so that it is also possible to record, in particular, the exothermic intensity (intensity of thermal radiation) and/or the time course of the exothermic reactions.

Recording and evaluation are preferably carried out by means of the sensor unit and an evaluation unit assigned thereto. It is, however, optionally also possible to carry out part of or the entire evaluation in said sensor unit.

Depending on the thermal radiation recorded, the sensor unit or its IR camera delivers measured signals which are edited by means of evaluation, taking into account, in particular, the time course or progress with time. The edited signals which represent, for example, the time course of the exothermicity or temperature of the individual reaction mixtures can preferably be displayed, printed out and/or output for further processing or storage, for example via a standardized interface or the like.

As already indicated in connection with the IR camera, the measured signals may also comprise additional information, in particular regarding optical parameters or changes in the reaction mixtures monitored. Preferably, this additional information is also evaluated and, accordingly, output as edited signals separately or together with the signals regarding exothermicity.

In principle, it is possible for the sensor unit to monitor the individual reaction chambers or the reaction mixtures contained therein sequentially, i.e. one after the other, individually or in groups, for example by moving the individual reaction chambers or groups of reaction chambers past said sensor unit accordingly. Preferably, however, the sensor unit is designed for monitoring a multiplicity of, in particular all, reaction chambers and the reaction mixtures contained therein simultaneously, i.e. at the same time, thus permitting identification and distinction of the thermal radiations emitted by the individual reaction mixtures. This spatial differentiation is, in particular, very readily possible using the IR camera provided with preference, since a camera is in principle provided and suitable for spatial distinction of various regions and thus of the different reaction mixtures.

The sensor unit and its IR camera deliver preferably electrical, in particular digital, measured signals which can be evaluated, as already explained. Accordingly, it is possible for data processors having appropriate software to carry out in a simple manner, in particular automated, evaluation, storage, display and the like.

It is, however, also possible for the sensor unit or the IR camera to generate records in the conventional sense, in particular photocurves, which are then used for identifying exothermic reaction processes.

In particular, a newly developed, automated and miniaturized system with a parallel array is described which may be used, as a highly sensitive array, for determining exothermic reaction processes such as polymerizations, addition reactions, condensation reactions, degradation reactions, etc., and of all reactions or complex reaction processes in which an exothermic reaction dominates. Due to the possibility of studying a multiplicity of samples or reaction processes for exothermicity within a very short time, the system of the invention is occasionally also referred to synonymously as “high scan thermo-array” below.

Preferably, the system is constructed as stand alone system from three combined work stations, with additional integration of a metering system. In the exemplary embodiment, the system comprises an IR camera (e.g. Thermoscan™ SC 500 IR camera from FLIR), a “Multidrop” system (e.g. Multidrop 384 from Labsystems), a thermomixer (e.g. Thermomixer comfort from Eppendorf) and an eight-channel pipetting system (e.g. metering device MicroLab SD from Hamilton).

The multidrop and thermomixer systems are designed, in particular, for the use of microtiter plates having different numbers of wells so that it is possible to process a multiplicity of samples in parallel at the same time.

The exothermicity of the individual microreactions in the reaction chambers (Wells) is followed preferably on line and visualized on a display. Appropriate software makes possible the quantitative evaluation of the change in temperature inside the wells or the reaction mixtures therein as a function of time.

Further advantages, properties, aspects and features of the present invention arise from the following description of a preferred exemplary embodiment illustrated in the drawing, in which:

FIG. 1 depicts a diagrammatic representation of a system of the invention; and

FIG. 2 depicts a diagram of the time course of the temperature of various reaction mixtures.

FIG. 1 depicts a system of the invention 1 (“high scan thermo-array”) for monitoring chemical reaction processes, in particular for recording exothermic chemical reaction processes. In principle, it is also possible for the system 1 to be intended only for recording exothermicity in general, for example of exceeding a threshold, in particular of a predetermined temperature. Preferably, the system 1 of the invention is intended for recording the course or progress, with regard to time and temperature, of at least one chemical reaction, in particular of a multiplicity of chemical reactions.

In the exemplary illustration, the system 1 has a reaction unit 2 with a multiplicity of spatially separated reaction chambers 3 for receiving reaction mixtures 4. FIG. 1 depicts the reaction unit 2 in a diagrammatic section. In particular, the reaction unit 2 extends also perpendicularly to the plane of the drawing, the reaction chambers 3 being arranged in particular side by side and one after the other in rows and being, for example, open at the top, as illustrated.

The system 1 has at least one metering unit 5, indicated diagrammatically in FIG. 1, for charging the reaction chambers 3. The metering unit 5 may be used for supplying the reaction chambers 3 with reaction components 6, 7. Depending on the design, the reaction components 6, 7 can be supplied to a reaction chamber 3 simultaneously or successively. Moreover, depending on the design, the metering unit 5 can charge individual reaction chambers 3 successively or a plurality of or all reaction chambers 3 simultaneously.

The reaction components 6, 7 may preferably be mixed only in the particular reaction chamber 3 or, if required, also beforehand.

The reaction components 6, 7 or reaction mixtures 4 are of course supplied in the desired amounts, in particular with different ratios, in order to be able to assay, for example, different reaction mixtures 4 or to record and evaluate the behavior thereof. It is of course also possible to supply various or further reaction components 6, 7 to the individual reaction chambers 3 to form completely different reaction mixtures 4. With respect to this, too, reference is made in particular to the “screening” known from the prior art, in particular high throughput screening (HTS).

It is essential that the system 1 has at least one sensor unit 8 which can detect thermal radiation 9 emitted from the reaction mixtures 4, i.e. which is sensitive to thermal radiation. The thermal radiation 9 is infrared (IR) radiation.

The sensor unit 8 is assigned to the reaction unit 2 in such a way that it is possible to record the thermal radiation 9 in the manner desired.

In principle, the sensor unit 8 may be designed in such a way that in each case only a single reaction chamber 3 can be monitored or the thermal radiation 9 of a reaction mixture 4 contained therein can be recorded. However, preference is given to designing the sensor unit 8 in such a way that it is possible to monitor a plurality of, in particular all, reaction chambers 3 or to record the thermal radiations 9 of reaction mixtures 4 contained therein simultaneously or in parallel.

If the sensor unit 8 does not monitor simultaneously all reaction chambers 3 of the reaction unit 2, preference is given to providing a plurality of sensor units 8 (not shown) for overall complete monitoring of all reaction chambers 3. Alternatively or additionally, it is possible to move the sensor units (EM) 8 and the reaction unit 2 in relation to one another in such a way that the reaction chambers 3 can be monitored successively, either individually or in groups.

In principle, the sensor unit 8 may be arranged directly adjacent to or close to reaction chambers 3 to be monitored. In the exemplary illustration, however, the sensor unit 8 is preferably arranged at a distance above the reaction unit 2 and the reaction chambers 3, said sensor unit 8 enabling all reaction chambers 3 to be monitored simultaneously.

In the preferred and illustrated exemplary embodiment, the sensor unit 8 comprises an infrared (IR) camera 10. Accordingly, it is simultaneously possible to monitor simultaneously a multiplicity of, in particular all, reaction chambers 3 or reaction mixtures 4 contained therein for exothermicity or thermal radiation 9.

If required, the sensor unit 8 or camera 10 may also be sensitive additionally in the ultraviolet or, in particular, visible wavelength range and, accordingly, may provide additional information about reaction processes or reaction mixtures 4, if required.

The sensor unit 8 or camera 10 is preferably designed so as to provide electrical measured signals or thermal radiation data which are evaluated according to requirements. To this end, in particular, in the exemplary illustration an evaluation unit 11 is connected directly to the sensor unit 8 or camera 10. However, the evaluation 8 may, if required, also be carried out partially or completely already in the sensor unit 8 or camera 10.

The evaluation unit 11 consists in particular of an evaluation program which is not explained in more detail here and which runs on a computer, microprocessor or the like. Evaluation or processing of the data is thus carried out with the aid of computers. It is, however, also possible to evaluate the measured signals or thermal radiation data independently of the system 1, for example subsequently.

The measured signals or thermal radiation data include of course also the necessary information in order to be able to relate the thermal radiations 9 recorded and the temperatures corresponding thereto to the particular reaction chambers 3 and thus to the particular reaction mixtures 4.

The measured signals or thermal radiation data provided by the sensor unit 8 or camera 10 may be stored intermediately, if required, and evaluated only at a later time. However, preference is given to carrying out a continuous evaluation, recording, in particular, also the time course of the thermal radiation or temperature of the individual reaction mixtures 4, i.e. the particular exothermic course of the reaction, as FIG. 2 illustrates, by way of example, for three different reaction processes. The courses of the reactions are, for example, continuously stored, printed out and/or displayed or are output to devices (not shown) for further processing, for example via an interface (not shown).

In particular, the evaluation records the temperature or a value proportional thereto of the reaction mixture 4 monitored in each case. The thermal radiation 9 recorded by the sensor unit 8 or camera 10, which is in particular intensity data, can be converted optionally already in the sensor unit 8 or in the subsequent evaluation. In particular, an appropriate calibration is possible or provided for. The conversion may be carried out, for example, by means of appropriate conversion parameters, value tables, interpolation or the like.

To correlate the times of the measured signals or thermal radiation data provided by the sensor unit 8 or camera 10, the system 1 has, in particular, a time base 12 or the like. It is, however, also possible to use for this, if required, instead of a separate time base 12, the internal clock of a computer or of another unit carrying out the evaluation and constituting, in particular, the evaluation unit 11.

The reaction unit 2 is preferably designed as a microtiter plate. Said reaction unit 2 has in particular reaction chambers 3 in the form of wells 13 which are in each case separated from one another by bridges 14 or the like. The reaction chambers 3 are preferably open at the top. However, if required, the reaction chambers 3 may also be sealed; they may, in particular, be sealable by a lid or the like (not shown) which reacts through thermal radiation 9.

FIG. 1 depicts the system 1 of the invention only diagrammatically. The reaction components 6, 7 can be supplied, for example, via lines or channels 15, 16 of the metering unit. Depending on the design, the metering unit 5 may have one or multiple channels, it being possible, for example, for the same reaction component 6 or 7 to be supplied to a plurality of reaction chambers 3 at the same time and/or for at least two different reaction components 6, 7 to be supplied to at least one reaction chamber 3 at the same time.

The system 1 preferably has a mixing unit 17 assigned to the reaction unit 2. The mixing unit 17 may, for example, agitate or shake the reaction unit 2 or the reaction chambers 3 thereof and/or cause an ultrasound action, for example by means of an ultrasound converter or the like, not shown. Alternatively or in addition, the mixing unit 17 may also have at least one stirrer 18, preferably a plurality of stirrers 18, assigned in each case to a reaction chamber 3. In particular, the stirrers 18, if provided, may be powered by an electrical drive 19.

It is essential that the mixing unit 17 readily mixes the reaction mixtures 4 or the reaction components 6, 7 thereof contained in the reaction chambers 3.

Furthermore, the system 1 preferably has a heating unit 20, for example in the form of a hotplate, a heating coil or an infrared heater, which is assigned to the reaction unit 2. The heating unit 20 is used for warming or heating of the reaction mixtures 4 contained in the reaction chambers 3, should this be desired.

The system 1 of the invention preferably has a control unit 21 which makes possible, in particular, an automated process, i.e., in particular, automated screening of a multiplicity of reaction mixtures 4 and thermal monitoring thereof. The control unit 21 is used in particular for controlling the metering unit 5, the evaluation unit 11 with the assigned sensor unit 8 or camera 10, the mixing unit 17 and/or the heating unit 20, as indicated by the dashed lines.

In the exemplary illustration, the evaluation unit 11 and the time base 12 are integrated in the control unit 21, although this is not absolutely necessary. Rather it is also possible for the evaluation unit 11 to consist, for example, of a separate computer or the like.

In the exemplary illustration, the above-described components or parts of the system 1 of the proposed invention preferably constitute an apparatus. Optionally, however, they may also be apparatuses which are at least partially separated or independent of one another.

To display the thermal reaction profiles, a display unit 22 is preferably assigned to the system 1. Said display unit 22, in particular a screen or the like, is, for example, directly connected to the evaluation unit 11 or to the control unit 21.

It should be mentioned that the evaluation can also be switched to different modes, if required. It is possible, for example, to switch between continuous monitoring of the thermal reaction processes and a function for warning or identification of a predeterminable temperature being exceeded.

Preference is given to displaying the recorded thermal processes continuously on the display unit 22, for example in the form of a diagram according to FIG. 2.

The present invention is illustrated by the following exemplary embodiment, but without being emitted thereto. Further embodiments, modifications and variations of the present invention are readily familiar to the skilled worker when reading the present description, without him leaving the scope of the present invention.

The method for evaluating exothermic reaction processes with the aid of the system 1 of the invention is described in detail using the following example of evaluating anaerobic adhesive formulations.

### Exemplary Embodiment

Preparation of anaerobic adhesive formulations comprising from ten to up to fifteen different reactive and inactive ingredients

Of importance for bonding are in particular monomers, for example methacrylates, initiators such as hydroperoxides, accelerators such as sulfonylamides and reducing agents such as, for example, tertiary amines. Part of these reactive components 6, 7 are supplied, where appropriate, in diluted form by means of a metering system 5 which charges the wells 3 of a 96-well microtiter plate. The metering system 5 used is a multichannel pipetting system (Hamilton MicroLab SD) which is distinguished by managing various liquids in parallel. In this way it is possible to transfer substances from a number of starting vessels to a number of target vessels.

In the case of the above-described array it is sufficient for each well to contain between from 10 to 100 μl, preferably 20 to 50 μl, of the reaction mixture.

Charging the individual wells of the microtiter plate with the microamounts of the reactants or the composition of the individual formulations is controlled via a software program and carried out in the high scan thermo-array of the invention. The formulations are homogenized by means of a thermomixer (Thermomixer comfort from Eppendorf). This is followed in the example described by starting the exothermic polymerization process by metering in from 1 to 10 μl of a metal salt solution. It is important here that all 96 wells are charged in no more than five seconds. Another homogenization is followed by the exothermic process which is IR-thermocurveically recorded for each well and visualized on a display.

FIG. 2 illustrates the thermocurveic profile of three selected reaction samples. FIG. 2 depicts, by way of example, a diagram of various reaction processes. Curve 23, for example, corresponds to a quickly curing adhesive, curve 24 to a moderately quickly curing adhesive and curve 25 to a slowly curing adhesive. Accordingly, different temperature maxima and different curve profiles occur at different times. Accordingly, the curves 23, 24 and 25 correlate with different reaction mixtures 4 in different reaction chambers 3.

It is of course possible here to display or depict the data in different ways. For example, the thermal reaction profile for each reaction mixture 4 may, if required, be called up individually or a plurality of, or all, profiles may be depicted on top of one another or side by side, one below the other, or combined in another way. If required, they may also be depicted in another way, for example in the form of number tables, or evaluated further, for example by reduction to temperature maxima and time.

1. A system (1) for monitoring chemical reaction processes, in particular for recording exothermic chemical reaction processes, which contains a reaction unit (2) having a multiplicity of spatially separated reaction chambers (3) for receiving reaction mixtures (4) and contains a metering unit (5) for introducing reaction components (6, 7) of said reaction mixtures (4) into said reaction chambers (3),
characterized in that,
the system (1) has at least one sensor unit (8) sensitive to thermal radiation for recording the thermal radiation (9) emitted from reaction mixtures (4) present in said reaction chambers (3).

2. The system as claimed in claim 1, characterized in that the sensor unit (8) comprises an IR camera (10).

3. The system as claimed in claim 1 or 2, characterized in that the sensor unit (8) comprises an IR spectrometer.

4. The system as claimed in any of the preceding claims, characterized in that the sensor unit (8) is designed in such a way that the thermal radiations (9) of a plurality of, in particular of all, reaction mixtures (4) can be recorded simultaneously or that a multiplicity of, preferably all, reaction chambers (3) can be monitored simultaneously.

5. The system as claimed in any of the preceding claims, characterized in that the sensor unit (8) can output electrical measured signals or thermal radiation data, in particular in digital form.

6. The system as claimed in any of the preceding claims, characterized in that the system (1) is designed in such a way that the reaction processes can be monitored continuously, preferably all of them at the same time.

7. The system as claimed in any of the preceding claims, characterized in that the system (1) is designed in such a way that the time course of exothermic reaction processes and/or exceeding a threshold, in particular a temperature, and/or the time for a maximum thermal radiation or temperature to be reached can be recorded and, in particular, displayed.

8. The system as claimed in any of the preceding claims, characterized in that the system (1) has an evaluation unit (11) for evaluating the measured signals or thermal radiation data provided by the sensor unit (8).

9. The system as claimed in claim 8, characterized in that the evaluation unit (11) is connected directly to the sensor unit (8), with, in particular, said evaluation unit (11) controlling said sensor unit (8).

10. The system as claimed in claim 8 or 9, characterized in that the evaluation unit (11) is designed for editing and/or analyzing and/or displaying the exothermicity of chemical reactions of the reaction mixtures (4), in particular of the time courses.

11. The system as claimed in any of claims 8 to 10, characterized in that the evaluation unit (11) comprises a computer or microprocessor.

12. The system as claimed in any of the preceding claims, characterized in that an evaluation unit (11) or the system (1) has a time base (12) for time-correlated monitoring and, in particular, evaluation of the reaction processes.

13. The system as claimed in any of the preceding claims, characterized in that the reaction unit (2) is designed in a flat and/or plate-like form, the reaction chambers (3) being designed in particular as wells (13).

14. An apparatus as claimed in claim 13, characterized in that the wells (13) are spatially separated by bridges (14).

15. The system as claimed in any of the preceding claims, characterized in that the reaction unit (2) has at least 10, in particular at least 100 to 200, preferably up to 100 reaction chambers (3), in particular in the form of wells (13), and/or that said reaction chambers (3) have a volume of in each case from 5 to 100 μl, in particular 10 to 50 μl, preferably 10 to 20 μl, and/or are open at the top and can be sealed, where appropriate, and/or that said reaction chambers (3) preferably have circular and U-shaped horizontal and, respectively, vertical cross sections.

16. The system as claimed in any of the preceding claims, characterized in that the reaction unit (2) is composed of nonmetallic material, in particular plastic.

17. The system as claimed in any of the preceding claims, characterized in that the reaction unit (2) is designed as a microtiter plate.

18. The system as claimed in any of the preceding claims, characterized in that the metering unit (5) is designed as a single-channel or multichannel supply system for supplying, in particular in each case simultaneously, reaction components (6, 7) to the reaction chambers (3) and/or supplying said reaction components simultaneously to a plurality of reaction chambers (3), with preferably in each case a single channel (15, 16) being provided for supplying a single reaction component (6, 7).

19. The system as claimed in any of the preceding claims, characterized in that the system (1) has at least one further metering apparatus (5) so as to supply different reaction components (6, 7) independently of one another to the reaction chambers (3).

20. The system as claimed in any of the preceding claims, characterized in that the system (1) has a mixing unit (17) for mixing reaction mixtures (4) contained in the reaction chambers (3).

21. The system as claimed in claim 20, characterized in that the mixing unit (17) comprises an agitator or shaker which ensures intensive mixing of reaction mixtures (4) contained in the reaction chambers (3), in particular by movements back and forth and/or movements up and down, tumbling and/or rotating movements.

22. The system as claimed in claim 20 or 21, characterized in that the mixing unit (17) comprises a sonicator and/or stirrers (18) which is/are assigned in each case to a reaction chamber (3).

23. The system as claimed in any of the preceding claims, characterized in that the system (1) has a heating unit (20) for heating the reaction mixtures (4) contained in the reaction chambers (3).

24. The system as claimed in any of claims 20 to 22 and as claimed in claim 23, characterized in that the heating unit (20) is assigned to the mixing unit (17) and/or integrated in said mixing unit (17).

25. The system as claimed in any of the preceding claims, characterized in that the system (1) has a control unit (21) for automatic process control, in particular for controlling the metering unit (5), the sensor unit (8), a mixing unit (17), a heating unit (20) and/or an evaluation unit (11).

26. The system as claimed in any of the preceding claims, characterized in that the system (1) has a display unit (22), in particular a screen, with, in particular, said display unit (22) being connected to an evaluation unit (11) or a control unit (21) of said system (1).

27. The system as claimed in any of the preceding claims, characterized in that the system (1) can be used in automated screening methods, in particular in high throughput screening.

28. The use of a system (1) as claimed in any of the preceding claims for monitoring and/or recording and/or regulating and/or controlling chemical reaction processes, in particular exothermic chemical reaction processes.

29. The use as claimed in claim 28 for monitoring and/or recording and/or regulating and/or controlling polymerization, polycondensation, polyaddition and degradation reactions, including biological or purely chemical degradation reactions.

30. The use as claimed in claim 28 or 29 in automated screening methods, in particular in high throughput screening.

31. The use as claimed in any of claims 28 to 30 for materials testing, in particular for quality control.

32. The use as claimed in any of claims 28 to 31 for testing for active substances or active substance systems, in particular active substances and active substance systems formed under exothermic reaction conditions.

33. The use as claimed in any of claims 28 to 32 in the development of adhesive systems, in particular anaerobic adhesive formulations.

34. The use as claimed in any of claims 28 to 33 for process regulation and/or for regulating and/or recording the course of the process.

35. The use of a sensor unit (8) sensitive to thermal radiation,
characterized in that,
said sensor unit (8) monitors and/or regulates chemical reaction processes of reaction mixtures (4) with respect to exothermicity by recording the thermal radiation (9) emitted by said reaction mixtures (4).

36. The use as claimed in claim 35, characterized in that a sensor unit (8) is used which, in particular, has an IR camera (10) in order to monitor a plurality of, in particular all, reaction processes at the same time.

37. The use as claimed in any of claims 28 to 36, characterized in that the time course of the exothermicity of the reaction processes is detected and, in particular, displayed.

38. A method for monitoring a multiplicity of chemical reaction mixtures (4), in which method individual reaction components (6, 7) of said reaction mixtures (4) are combined and preferably, where appropriate, homogeneously mixed,
characterized in that,
thermal radiation (9) arising is recorded in order to detect the exothermicity of the reaction mixtures (4) emitting said thermal radiation (9).

39. The method as claimed in claim 38, characterized in that the thermal radiation (9) is recorded by means of an IR camera (10).

40. The method as claimed in claim 38 or 39, characterized in that the thermal radiation (9) is recorded in a continuous and, in particular, time-correlated manner and is, in particular, displayed, stored or printed out.

41. The method as claimed in any of claims 38 to 40, characterized in that the thermal radiation (9) of a multiplicity of reaction mixtures (4) is recorded and evaluated simultaneously and independently of one another.
